(19) United States
(12) Patent Application Publication
(10) Pub. No.: US 2020/0253721 A1
(43) Pub. Date: Aug. 13, 2020

(54) SINGLE PIECE INTRA-OCULAR LENSES AND METHODS OF MANUFACTURE THEREOF

(71) Applicant: CLEARSIGHT, LLC, Aurora, CO (US)

(72) Inventors: Kevin Cuevas, Littleton, CO (US); Shravanthi Reddy, Goleta, CA (US); Khalid Mentak, San Ramon, CA (US); Kevin S. Harris, Denver, CO (US)

(21) Appl. No.: 16/649,184

(22) PCT Filed: Sep. 20, 2018
(86) PCT No.: PCT/US2018/051967

§ 371 Date: Mar. 20, 2020

Related U.S. Application Data
(60) Provisional application No. 62/560,860, filed on Sep. 20, 2017.

(51) Int. Cl. A61F 2/16

(52) U.S. Cl. CPC A61F 2/16; A61F 2250/0025; A61F 2240/001; A61F 2250/0053; A61F 2002/1681; A61F 2250/0051

(57) ABSTRACT

Disclosed herein is an intraocular lens implantable in an eye comprising an optical portion adapted for placement in the lens capsule of the eye and for directing light toward the retina of the eye; an annular ring having an outer diameter and an inner diameter; and at least one elongated fixation member coupled to said optical portion for use in fixing said intraocular lens in the eye; where the outer diameter of the annular ring contacts the elongated fixation member and where the inner diameter of the annular ring contacts the optical portion of the intraocular lens; and where the entire intraocular lens is a single monolithic piece.

### CROSS REFERENCE TO RELATED APPLICATIONS

This application claims the benefit of U.S. Application No. 62/560,860, filed on Sep. 20, 2017, which is incorporated herein by reference in its entirety.

### BACKGROUND

This disclosure relates to single piece intra-ocular lenses and to methods of manufacture thereof.

Visually impairing cataracts are the leading cause of preventable blindness in the world. Presently, the only known treatment for cataracts is the surgical removal of the opacified lens of the affected eye and replacement with an artificial intraocular lens (“IOL”). Technological advances in cataract surgery with IOL implantation have made cataract surgery among the most effective surgical procedures.

FIGS. 1 and 2 show a top view and a cross-sectional view of a phakic eye (1). The most common technique of cataract surgery may be extracapsular cataract extraction (“ECCE”) which involves the creation of an incision (42) near the outer edge of the cornea (2) and a circular opening (44) (shown in FIGS. 3 and 4) in the anterior lens capsule (43) (also herein referred to as the “anterior capsule”) through which the opacified lens (3) can be removed from the lens capsule (45) (also referred to as the “capsular bag”). FIGS. 3 and 4 show a top view and a cross-sectional view of a pseudophakic eye (4). The lens capsule (43), anchored to the ciliary body (6) through the zonular fibers (7), can be left substantially intact. The IOL (8) can then be placed within the lens capsule (43) through the circular opening (44) in the anterior capsule (43). The IOL (8) can be acted on by zonular forces exerted on the outer circumference of the lens capsule (45) which establishes the location of the IOL (8) within the lens capsule (45). The intact posterior capsule (5) acts as a barrier to the vitreous humor (9) within the posterior segment of the eye.

The most frequent complication to ECCE and other methods of cataract surgery can be opacification of the posterior capsule (5). Posterior capsule opacification (“PCO”) results from the migration of residual lens epithelial cells (“LEC”) between the IOL (8) and the surface of the posterior capsule (5) subsequent to cataract surgery. The residual LECs once located between the IOL (8) and the surface of the posterior capsule (5) can proliferate leading to clouding of the normally clear posterior capsule (5). Clouding of the posterior capsule (5) can decrease visual acuity if the opacification occurs within the visual axis (21).

Visually significant PCO requires an additional surgery to clear the visual axis of the eye. Presently, the most widely utilized procedure to clear the visual axis of PCO may be Neodymium: Yttrium-Aluminum-Garnet (“Nd:YAG”) laser capsulotomy. However, there may be substantial problems with this procedure such as IOL damage, postoperative intraocular pressure spikes, vitreous floaters, cystoid macular edema, retinal detachment, and IOL subluxation, or the like. Additionally, pediatric patients can be difficult to treat and a delay in treatment can lead to irreversible amblyopia. Many underdeveloped countries do not have access to a Nd:YAG laser and the cost can be prohibitive.

Prevention or inhibition of PCO mechanisms fall into two broad categories: mechanical and pharmacological. Mechanical mechanisms to inhibit PCO have primarily focused on configuration of the IOL (8). Configuring the IOL to include a sharp posterior edge may provide a structural barrier to the migration of residual LECs between the IOL and the surface of the posterior capsule (5). Cleary et al., Effect of Square-edged Intraocular Lenses on Neodymium: YAG Laser Capsulotomy Rates in the United States, J. Cataract & Refractive Surgery, Vol. 13, p. 1899 (November 2007). However, while introduction of square edged IOLs appears to have reduced incidence of PCO, a review of Medicare claims data from 1993 to 2003 evidences that the number of laser capsulotomies performed in the United States to treat PCO in recipients of square edged IOL remains substantial.

Pharmacological mechanisms have been proposed as a way to inhibit or prevent PCO. The effect of topical treatment with nonsteroidal anti-inflammatory drugs (“NSAIDs”) such as diclofenac and indomethacin after phacoemulsification do not appear to inhibit PCO. Inan et al., Effect of Diclofenac on Prevention of Posterior Capsule Opacification in Human Eyes, Can J Ophthalmol, 41; 624-629 (2006). Additionally, the majority of pharmacological agents tested in vitro for inhibition of migration and proliferation of LECs are antimetabolites and antimitotics which have not been used clinically because of their toxic side effects. Inan U U, Ozturk F, Kaynak S, et al. Prevention of Posterior Capsule Opacification by Intraoperative Single-dose Pharmacologic Agents, J Cataract Refract Surg, 27: 1079-87(2001); Inan U U, Ozturk F, Kaynak S. Ilker S S, Ozer E, Güler, Prevention of Posterior Capsule Opacification by Retinoic Acid and Mitomycin, Graefes Arch Clin Exp Ophthalmol 239: 693-7(2001); Cortina P, Gomez-Lechon M J, Navea A, Menezo J L, Terencio M C, Diaz-Llopis, M, Diclofenac Sodium and Cyclosporine A Inhibit Human Lens Epithelial Cell Proliferation in Culture, Greaefes Arch Clin Exp Ophthalmol 235: 180-5(1997); Ismail M M, Alio J L, Ruiz Moreno J M, Prevention of Secondary Cataract by Antimitotic Drugs: Experimental Study, Ophthalmic Res, 28: 64-9 (1996); Emery J., Capsular Opacification After Cataract Surgery, Curr Opin Ophthalmol 9: 60-5 (1998); Hartmann C, Wiedemann P, Gothe K, Weller M Heimann K, Prevention of Secondary Cataract by Intracapsular Administration of the Antibiotic Daunomycin, Ophthalmologic, 4:102-6 (1990).

Also, available is a sealed capsule irrigation device which functions to allow selective irrigation of the lens capsule with LEC inhibiting pharmacologic agents. Maloof A J, Neilson G, Milverton E J, Pandy S K, Selective and specific targeting of lens epithelial cells during cataract surgery using sealed-capsule irrigation, J Cataract Refract Surg, 29:1566-68 (2003). It is not clear, however, that use of the device can be reduced to routine practice. Problems relating to incomplete seal of the lens capsule (45) resulting in leakage of potentially toxic chemicals into the anterior chamber (46) of the eye, rupture of the lens capsule (45) during manipulation of the irrigation device, difficulty in assessing kill of LECs within the lens capsule and an increase in the duration of routine cataract surgery limit the usefulness of the irrigation device.

Another prominent problem with routine cataract surgery and other surgical procedures such as retinal surgery, cornea transplant surgery, glaucoma surgery, or the like, can be postoperative administration of antibiotics to prevent endophthalmitis. Topical antibiotic and anti-inflammatory eye drops represent the mainstay of drug delivery for intraocular surgery. However, there has yet to be a prospective randomized study showing that topical antibiotics prevent endophthalmitis. Also, because the human cornea acts as a natural barrier to biologic and chemical insults, intraocular bioavailability usually requires frequent dosing regimens for each medication. Topical drops can be difficult for young and elderly patients and the drop schedule can be cumbersome and confusing particularly when, following surgery, each eye is on a different drop schedule. These difficulties can result in non-compliance with serious consequences such as endophthalmitis, glaucoma, and cystoid macular edema. Recent prospective studies supporting the use of intracameral antibiotic injections for prophylaxis of endophthalmitis have stirred debate regarding the risks associated with this method of antibiotic prophylaxis including the short duration of protective effect (possibly less than 24 hours), the introduction of potentially contaminated substances in the anterior chamber, endothelial cell toxicity, toxic anterior segment syndrome, dilutional and osmolarity errors during mixing, and the like. Also, the systemic administration of drugs for treatment of localized ocular conditions may not be preferred because of the inefficiency associated with indirect delivery of the drugs to a target organ.

### SUMMARY

Disclosed herein is an intraocular lens implantable in an eye comprising an optical portion adapted for placement in the lens capsule of the eye and for directing light toward the retina of the eye; an annular ring having an outer diameter and an inner diameter; and at least one elongated fixation member coupled to said optical portion for use in fixing said intraocular lens in the eye; where the outer diameter of the annular ring contacts the elongated fixation member and where the inner diameter of the annular ring contacts the optical portion of the intraocular lens; and where the entire intraocular lens is a single monolithic piece.

Disclosed herein too is a method comprising molding in one piece an intraocular lens comprising an optical portion adapted for placement in the lens capsule of the eye and for directing light toward the retina of the eye; an annular ring having an outer diameter and an inner diameter; and at least one elongated fixation member coupled to said optical portion for use in fixing said intraocular lens in the eye; where the outer diameter of the annular ring contacts the elongated fixation member and where the inner diameter of the annular ring contacts the optical portion of the intraocular lens; and where the entire intraocular lens is a single monolithic piece.

Disclosed herein too is a method comprising making an incision is made in an edge of a cornea of any eye; making a circular opening in an anterior portion of a lens capsule of the eye; and disposing an intraocular lens within the lens capsule through the circular opening in the anterior portion; where the intraocular lens comprises an optical portion adapted for placement in the lens capsule of the eye and for directing light toward the retina of the eye; an annular ring having an outer diameter and an inner diameter; and at least one elongated fixation member coupled to said optical portion for use in fixing said intraocular lens in the eye; where the outer diameter of the annular ring contacts the elongated fixation member and where the inner diameter of the annular ring contacts the optical portion of the intraocular lens; and where the entire intraocular lens is a single monolithic piece.

Disclosed herein too is a retaining cell for holding an intraocular lens comprising a back wall having an opening for accommodating an aperture of an intraocular lens; such that the opening and the aperture are aligned for directing light toward the retina of the eye; a sidewall that contacts the back wall along its circumference; and at least two lips that contact the side wall and protrude radially from a circumference of the side wall towards a center of the opening in the back wall; where a surface of the lip is parallel to a surface of the back wall creating a space therebetween for reversibly accepting an intraocular lens.

### BRIEF DESCRIPTION OF THE FIGURES

FIG. 1 is a top view of the phakic eye with the natural lens intact;

FIG. 2 is a cross-sectional view, taken along line 2-2 of FIG. 1, of the phakic eye with the natural lens intact;

FIG. 3 is a top view of the pseudophakic eye having the natural lens replaced with an IOL;

FIG. 4 is a cross-sectional view, taken along line 4-4 of FIG. 3, of the pseudophakic eye having the natural lens replaced with an IOL;

FIG. 5 depicts an exemplary embodiment of one side view of the IOL; the FIG. 5 also depicts the shape taken at sections AA, BB and CC;

FIG. 6 depicts an exemplary schematic isometric view of a manufactured IOL;

FIG. 7 depicts a schematic end-on view of an exemplary embodiment of the IOL;

FIG. 8 depicts an exemplary embodiment of a side view of the IOL that contains a membrane for supporting the optical portion;

FIG. 9 depicts portions of the annular ring that are patterned to prevent cells and fluid from intruding onto the optic and the field of view;

FIG. 10 depicts a circular pattern that is used on the annular ring to prevent cells and fluid from intruding onto the optic and the field of view;

FIG. 11 depicts another circular pattern that is used on the annular ring to prevent cells and fluid from intruding onto the optic and the field of view;

FIG. 12 depicts a retaining cell for containing an intraocular lens;

FIG. 13 depicts a retaining cell with the intraocular lens contained therein; and

FIG. 14 depicts one method for disposing the intraocular lens into a retaining cell while placed in the lenticular capsule.

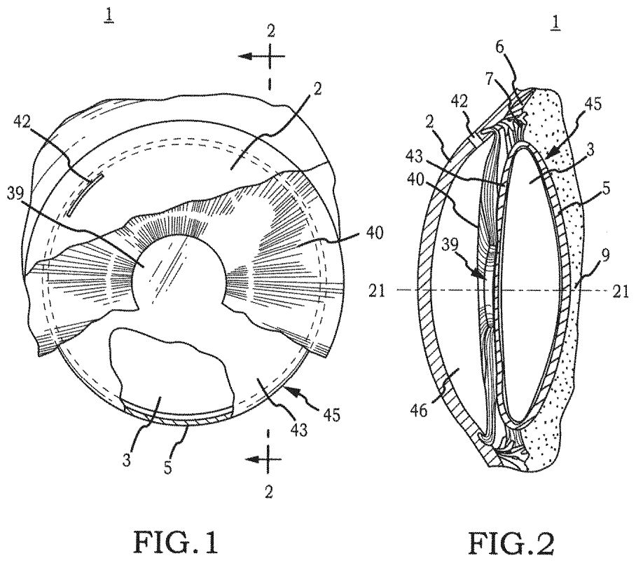

### DETAILED DESCRIPTION

Disclosed herein is a flexible, single piece intra-ocular lens (hereinafter “IOL”) that may be placed in the lens capsule (43) (see FIG. 2 and FIG. 4). The lens comprises a haptic and an optic that are formed into a single piece and that can be easily manipulated into position during surgery. The use of single piece lens reduces the time for implantation of the lens into the lens capsule during surgery. It also reduces the possibility of the separation of the optic from the haptic during storage or during or after surgery.

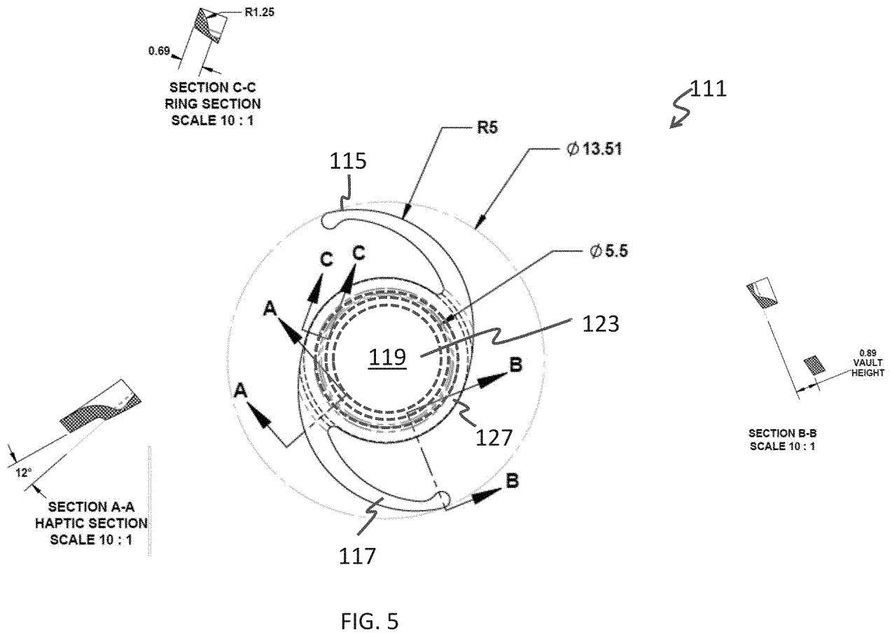

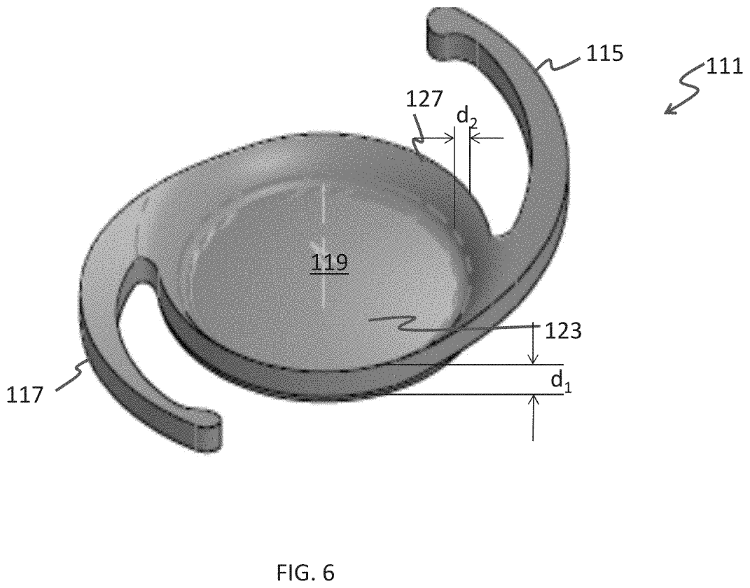

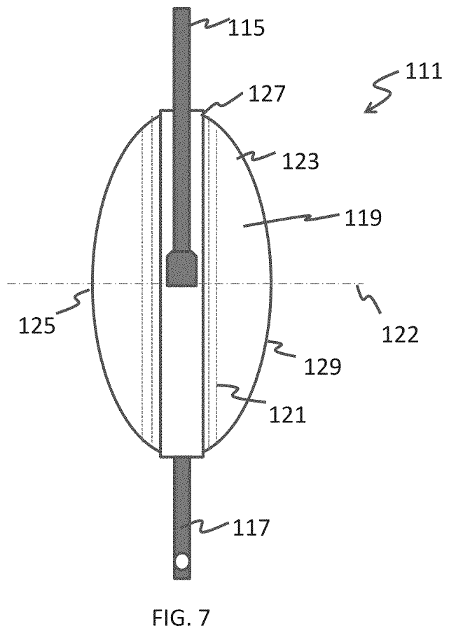

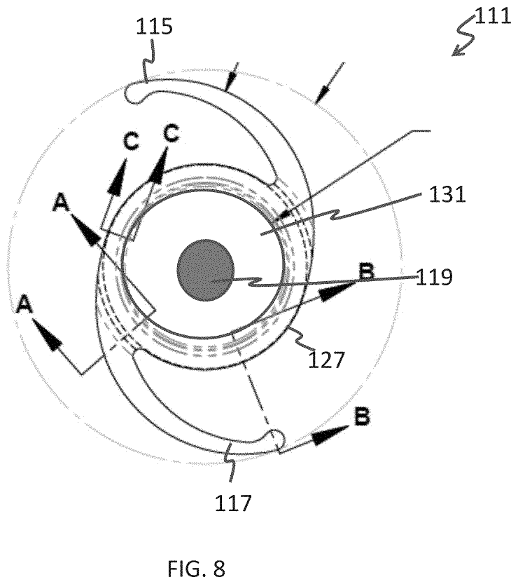

With reference now to the FIGS. 5, 6 and 7, a single piece IOL 111 comprises an optic 123 (for focusing light on or near the retina of the eye) and a haptic that is used to facilitate location of the IOL 111 in the lens capsule (not shown in the FIGS. 5-7). The FIG. 5 depicts an exemplary embodiment of one side view of the IOL, while the FIG. 6 depicts an exemplary schematic isometric view of a manufactured IOL 111. FIG. 7 depicts a schematic end-on view of an exemplary embodiment of the IOL 111. The haptic includes fixation members 115 and 117. In this embodiment, the optic 123 may be considered as including an optical portion 119 for focusing light on or near the retina of the eye and an optional cell barrier portion 121 circumscribing the optical portion and being incapable of focusing light on the retina. Optical axis 122 (See FIG. 7) passes through the center of optic 123 in a direction generally transverse to the plane of the optic. The terms optic 123 and optical portion 119 are used interchangeably herein. The optical portion 119 can include the entire optic 123 (See FIGS. 5, 6 and 7) or can only be a portion of the optic 123 (See FIG. 8).

With reference now to the FIG. 6, the optic 123 is circular in plan (when viewed from the top). FIG. 7 depicts the optic 123 as being biconvex; however, this is purely illustrative as other configurations and shapes may be employed. For example in the FIG. 7, opposing surfaces 125 and 129 of the optic may be either convex, flat or concave.

The entire IOL may be constructed of any of the commonly employed materials commonly used for rigid or flexible optics. It is desirable for these materials to be biocompatible. Examples of materials used for rigid optics include polymeric materials such as polycarbonate, polymethylmethacrylate, polyolefin copolymers, polystyrene, polyacrylate, polyetherimides, or a combination thereof.

Polymeric materials used in resiliently deformable optics may also be used to form the entire IOL, such as polysiloxanes, acrylic polymeric materials, hydrogel-forming polymeric materials, mixtures thereof, and the like.

In an embodiment, copolymers of polysiloxanes may be used to form the entire IOL. Such copolymers include polycarbonate-polysiloxane copolymers, polymethylmethacrylate-polysiloxane copolymers, polyetherimide-polysiloxane copolymers, polytetrafluoroethylene-polysiloxane copolymers, polyolefin-polysiloxane copolymers.

The fixation members 115 and 117 in this embodiment are generally C-shaped and are integral with the optic 123 via an annular ring 127. However, this is purely illustrative as the fixation members 115 and 117 may be of other configurations.

As may be seen in the FIG. 5, the outer diameter of the IOL 111, as defined by the fixation members is 13.5 millimeters or less, preferably 13 millimeters or less, and more preferably 12.5 millimeters or less.

The optic 123 has an anterior face 125, a posterior face 129 and a peripheral edge 127, which is in the form of an annular ring (hereinafter “annular ring 127”). In this embodiment, the faces 125 and 129 are convex and the peripheral edge 127 is cylindrical, but as indicated above, these shapes are shown only by way of example.

The annular ring 127 is circular in shape having an outer diameter that contacts the fixation members 115 and 117 and an inner diameter that contacts the optic 123. As seen in the FIG. 6, the difference in size between the inner diameter and the outer diameter (d₂) is 0.75 millimeters or less, preferably 0.7 millimeters or less, and more preferably 0.69 millimeters or less. The thickness (d₁) is 1 millimeter or less, preferably 0.95 millimeters or less, preferably 0.90 millimeters or less. The inner diameter transitions to the optic 123 via a smooth radial surface shown in sections BB and CC.

The optic 123 is designed to be placed in the lens capsule. The outer diameter of the annular ring 127 is 7 millimeters or less, preferably 6.5 millimeters or less. The annular ring 127 contacts the fixation members 115 and 117 on its outer radial surface. The annular ring 127 also contacts the optic 123 on its inner radial surface. The outer diameter of the optic 123 (which corresponds to the inner diameter of the annular ring 127) may be 6 mm or less, preferably 5.8 mm or less, and more preferably 5.5 mm or less. The optical portion 119 performs the normal function of the optic of an IOL, i.e., to appropriately focus light at or near the retina. The optical portion 119 may be monofocal or multifocal. The optical portion is generally optically transparent.

The optic 123 has an outer diameter of 6.5 millimeters or less, preferably 6 millimeters or less, preferably 5.8 millimeters or less, preferably 5.5 millimeters or less, and more preferably 5.0 millimeters or less.

The optional cell barrier portion 121 circumscribing the optical portion 119 is integral with the optical portion 119 and is scribed on at least one surface of the optical portion. The cell barrier portion 121 generally does not focus light on the retina of the eye and includes an irregularly configured structure or surface feature effective to inhibit, and preferably substantially prevent, cell growth or cell migration radially inwardly across the cell barrier portion.

In an embodiment, the cell barrier portion 121 includes a textured surface. The textures are detailed in U.S. patent application having Ser. No. 14/298,318 to Cuevas et al, the entire contents of which are hereby incorporated by reference.

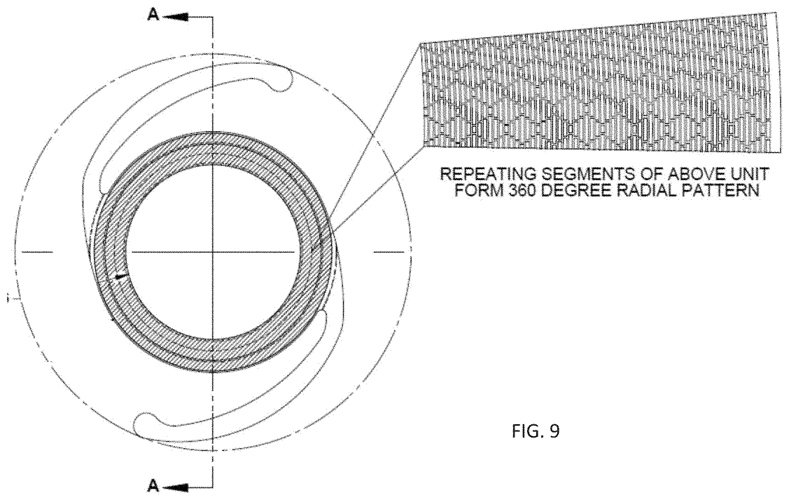

In an embodiment, one or more surfaces of the annular ring 127 may include a textured surface to prevent migration of epithelial cells into the optic 123 and into the field of view. The inner surface and the outer surface of the annular ring 127 may be textured with patterns shown in the FIGS. 9, 10 and 11. FIG. 9 depicts texturing disposed on inner surfaces of the annular ring. In an embodiment, texturing may be disposed on outer surfaces of the annular ring. Surfaces of the haptic may also be textured if desired.

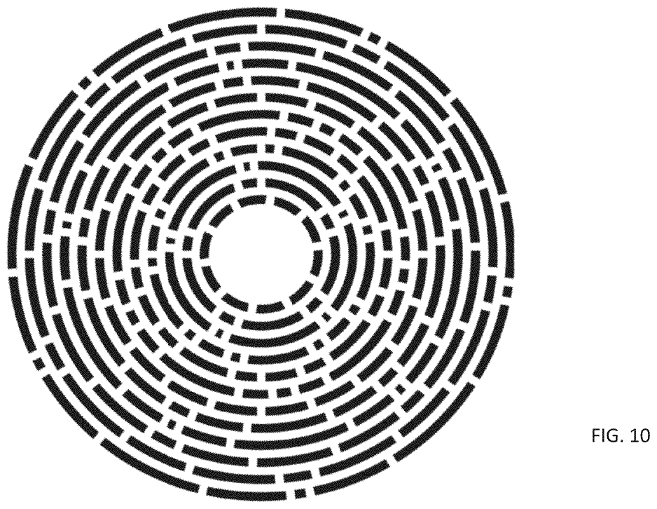

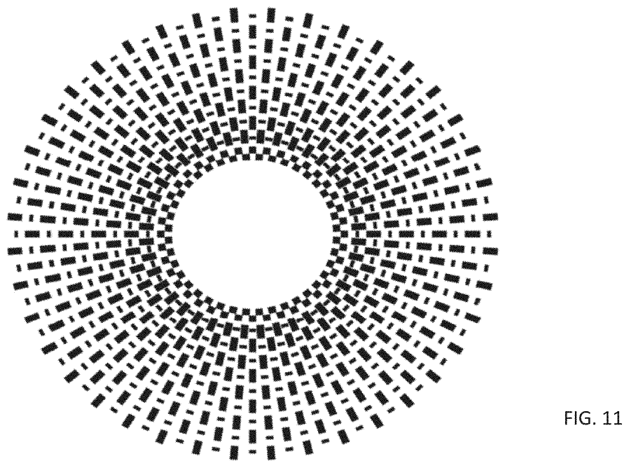

FIGS. 10 and 11 also depict different patterns that may be used to texture the surface of the annular ring. The texturing is detailed in U.S. Pat. Nos. 7,143,709, 7,650,848, 8,997,672, and 9,016,221 to Brennan et al., the entire contents of which are hereby incorporated by reference. The texturing is detailed below.

In one embodiment, the IOL 111 may have an annular membrane 131 that contacts the annular ring 127 on one circumference and the optical portion 119 of the optic 123 on an opposing circumference. This is depicted in the FIG. 8, where the membrane 131 has an outer circumference that contacts the inner surface of the annular ring 127. The membrane 131 has an inner circumference that is generally concentric with the outer circumference and that contacts the optical portion 119 of the optic 123.

The membrane is generally manufactured from a flexible polymer that is biocompatible. The opposing surfaces of the membrane are parallel to one another.

The entire IOL is manufactured in a single piece, i.e., it is monolithic and cannot be taken apart without damaging or destroying it. In an embodiment, the entire IOL comprises a single material, while in other embodiments, different portions of the IOL can comprise different materials, where the entire IOL exists in a single monolithic piece.

In one embodiment, in one method of manufacturing the IOL, a molten plastic is injected into a mold that has the requisite dimensions. The mold has the appropriate texturing in those portions so that texturing can be imparted to the IOL as depicted in the FIG. 5. Injection molding is preferred. Compression molding may also be used. Finishing operations such as lapping, grinding, surface polishing may be performed on the intraocular lens if desired.

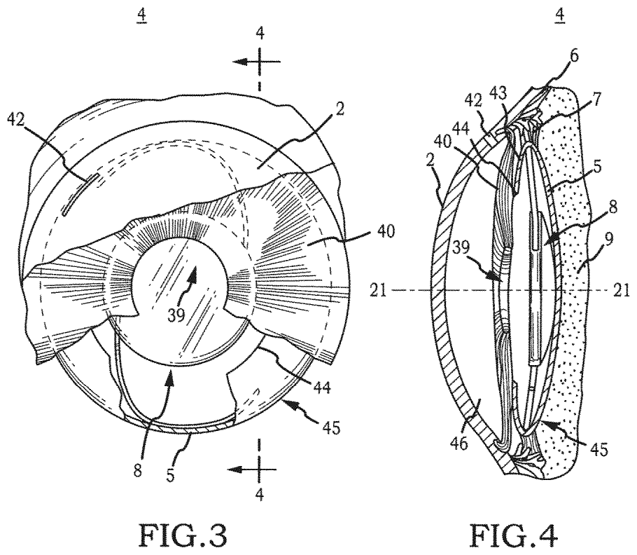

In an embodiment, in one method of using the IOL, as is already detailed in the FIGS. 3 and 4, an incision is made in the outer edge of the cornea 2 and a circular opening 44 in the anterior lens capsule 43 (also herein referred to as the “anterior capsule”) through which the opacified lens 3 can be removed from the lens capsule 45. FIGS. 3 and 4 show a top view and a cross-sectional view of a pseudophakic eye 4. The lens capsule 43, anchored to the ciliary body 6 through the zonular fibers 7, can be left substantially intact. The IOL 8 can then be placed within the lens capsule 43 through the circular opening 44 in the anterior capsule 43.

The surface texture disposed on the annular ring 127 can comprise a plurality of patterns. In one embodiment, the pattern generally has some features that are of the order of a few nanometers to several hundreds of millimeters in size. Each pattern is defined by a plurality of spaced apart features attached to or projected into the surface of the annular ring 127. The plurality of features on the surface each has at least one neighboring feature that has a substantially different geometry or a substantially different size. The average spacing between adjacent features on the surface texture is between about 1 nanometer to about 1 millimeter in at least a portion of the surface. The surface of the annular ring 127 may be planar, curved, or include portions that are planar combined with other portions that are curved.

In one embodiment, when the surface texture is viewed in a first direction, the plurality of spaced apart features is represented by a periodic function. In another embodiment, the plurality of spaced apart features forms a pattern. Each pattern is separated from a neighboring pattern by a pathway that has a periodicity to it. The periodicity of this pathway may be sinusoidal.

In one embodiment, the surface texture can comprise a pattern that comprises a plurality of spaced features. The spaced features are arranged in a plurality of groupings. The groupings of features comprise repeat units that can be repeated laterally and longitudinally across the surface. The spaced features within a grouping are spaced apart at an average distance of about 1 nanometer to about 500 micrometers, preferably at least 1 nanometer to about 10 micrometers. Each spaced feature has a surface that is substantially parallel to a surface on a neighboring feature. Each feature is separated from a neighboring feature and the groupings of features are arranged with respect to one another so as to define a tortuous pathway.

In yet another embodiment, the surface texture comprises a plurality of spaced features. The features are arranged in a plurality of groupings such that the groupings of features comprise repeat units. The spaced features within a grouping are spaced apart at an average distance of about 1 nanometer to about 500 micrometers, preferably about 1 nanometer to about 10 micrometers. The groupings of features are arranged with respect to one another so as to define a tortuous pathway where a tangent to the tortuous pathway intersects with a spaced feature. The spaced feature is different in geometry (shape or size) from each nearest neighbor and is not in contact with the nearest neighbor.

In yet another embodiment, the surface texture has a topography that comprises a pattern defined by a plurality of spaced apart features attached to or projected into a base surface of the annular ring 127. The plurality of features comprise at least one feature having a substantially different geometry, wherein neighboring patterns share a common feature, the plurality of spaced apart features having at least one dimension that is about 1 nanometer to about 1,000 micrometers. The neighboring spaced apart features can be spaced apart by a distance of about 5 nanometers to about 500 micrometers, specifically about 10 nanometers to about 100 micrometers, specifically about 1 micrometer to about 50 micrometers, and more specifically about 2 micrometers to about 25 micrometers.

In yet another embodiment, the surface texture comprises a plurality of spaced features; the features being arranged in a plurality of groupings; the groupings of features comprising repeat units; the spaced features within a grouping being spaced apart at an average distance of about 1 nanometer to about 200 millimeters. The groupings of features are arranged with respect to one another so as to define a tortuous path. In one embodiment, a tangent to the tortuous path intersects with at least one of the features.

In one embodiment, when viewed in a second direction, the pathway between the features may be non-linear and non-sinusoidal. In other words, the pathway can be non-linear and aperiodic. In another embodiment, the pathway between the features may be linear but of a varying thickness. The plurality of spaced features may be projected outwards from a surface or projected into the surface. In one embodiment, the plurality of spaced features may have the same chemical composition as the surface. In another embodiment, the plurality of spaced features may have a different chemical composition from the surface.

The tortuous pathway may be represented by a periodic function. The periodic functions may be different for each tortuous pathway. In one embodiment, the patterns can be separated from one another by tortuous pathways that can be represented by two or more periodic functions. The periodic functions may comprise a sinusoidal wave. In an exemplary embodiment, the periodic function may comprise two or more sinusoidal waves.

In another embodiment, when a plurality of different tortuous pathways are represented by a plurality of periodic functions respectively, the respective periodic functions may be separated by a fixed phase difference. In yet another embodiment, when a plurality of different tortuous pathways are represented by a plurality of periodic functions respectively, the respective periodic functions may be separated by a variable phase difference.

In another embodiment, the topography of the surface texture 104A, 104B has an average roughness factor (R) of from 2 to 50.

In one embodiment, each feature of a pattern has at least one neighboring feature that has a different geometry (e.g., size or shape). A feature of a pattern is a single element. Each feature of a pattern has at least 2, 3, 4, 5, or 6 neighboring features that have a different geometry from the feature. In one embodiment, there are at least 2 or more different features that form the pattern. In another embodiment, there are at least 3 or more different features that form the pattern. In yet another embodiment, there are at least 4 or more different features that form the pattern. In yet another embodiment, there are at least 5 or more different features that form the pattern.

In another embodiment, at least two identical features of the pattern have at least one neighboring feature that has a different geometry (e.g., size or shape). A feature of a pattern is a single element. In one embodiment, two identical features of the pattern have at least 2, 3, 4, 5, or 6 neighboring features that have a different geometry from the identical features. In another embodiment, three identical features of the pattern have at least 2, 3, 4, 5, or 6 neighboring features that have a different geometry from the identical features.

In another embodiment, each pattern has at least one or more neighboring patterns that have a different size or shape. In other words, a first pattern can have a second neighboring pattern that while comprising the same features as the first pattern can have a different shape from the first pattern. In yet another embodiment, each pattern has at least two or more neighboring patterns that have a different size or shape. In yet another embodiment, each pattern has at least three or more neighboring patterns that have a different size or shape. In yet another embodiment, each pattern has at least four or more neighboring patterns that have a different size or shape.

The texturing on the surfaces of the annular ring 127 may be represented by nomenclature. One example of the nomenclature adopted here may be represented by +XSKY×Z and should deciphered as follows: The +X indicates the height of the texture above the base surface of the clamp while the SK refers to a Sharklet pattern depicted and described in U.S. Pat. No. 7,143,709 B2 to Brennan et al., and patent application having Ser. No. 12/550,870 to Brennan et al. FIG. 2A in this document represents the Sharklet pattern. The negative sign (−) preceding the X would indicate that the texture is below the base surface. The Y in XKY×stands for the width of each feature in the pattern while the second Z stands for the spacing between the features in the pattern.

In an embodiment, the surface texture disposed on the annular ring 127 as shown in FIGS. 9, 10 and 11. FIGS. 10 and 11 depict an embodiment where the elements of the pattern are arranged in a circumferential direction on the annular ring 127. FIG. 10 depicts one embodiment where the elements of the pattern are arranged to be parallel with one another in the circumferential direction. In other words the elements of the pattern are concentric about the center point of the pattern. FIG. 11 depicts an embodiment where the elements of the pattern are arranged in a radial direction. These patterns can be used to control the flow of fluids from the center of the texture to the outer circumference and vice versa.

In one embodiment, the elements of the pattern are arranged to be parallel with one another in the circumferential direction along the surface of the annular ring 127 such that the spacing of elements relative to one another forms a continuous pattern along the circumference of the inner surface. In another embodiment, the elements of the pattern are arranged to be parallel with one another in the circumferential direction along the surface of the annular ring 127 such that the spacing of each element relative to one another forms a discontinuous pattern along the circumference of the inner surface with gaps in between groupings of elements. Any number of elements may be grouped together in between the gaps, e.g., 3, 5 or 7 elements.

In one embodiment, the intraocular lens 111 (detailed in the FIGS. 5-7) may be disposed in a retaining cell prior to being disposed in the lens capsule. The holding cell is designed to be placed in the lens capsule with the intraocular lens 111 contained therein. The intraocular lens 111 may be replaced in the lens capsule whenever desired. In other words, an older intraocular lens 111 may be removed from the lens capsule and replaced with a new one whenever desired or when necessary.

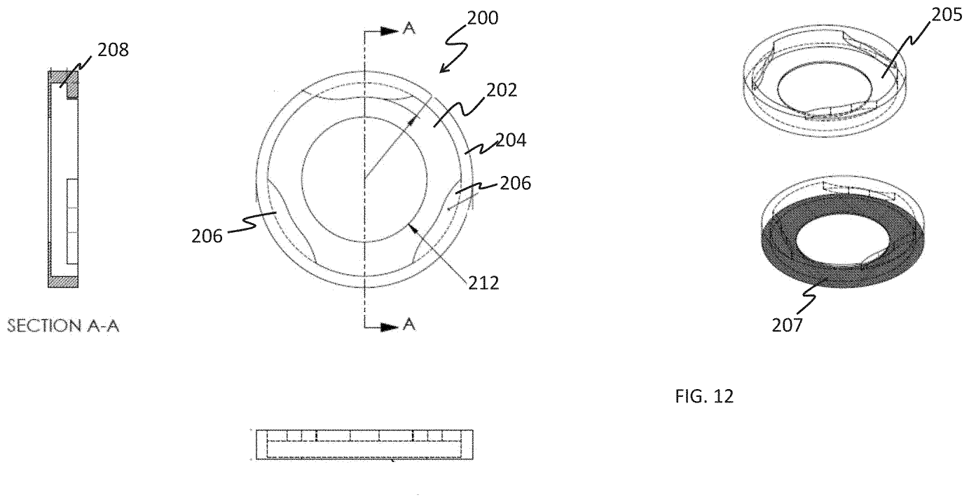

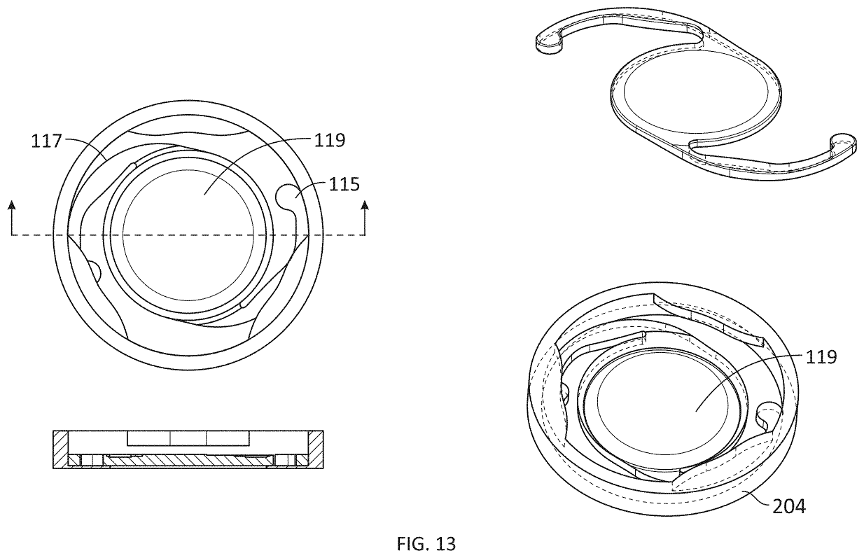

With reference now to the FIG. 12, the retaining cell 200 comprises a circular intraocular lens holder that comprises a back wall 202, a side wall 204 and two or more lips 206 for holding the intraocular lens 111 in place when the retaining cell 200 is placed in the lens capsule. The intraocular lens 111 is held in position in a space 208 between the lips 206 and the back wall 202. The FIG. 13 displays one exemplary embodiment, where the intraocular lens 111 is held in position between the lip 206 and the back wall 202 before bring placed in the lens capsule.

With reference now to the FIGS. 12 and 13, the back wall 202 has a smooth inner surface 205 opposed to a textured outer surface 207. The textured outer surface may contain the texture detailed earlier in the FIGS. 9, 10 and 11. The back wall 202 has at its center an opening which corresponds to the aperture 119 (See FIGS. 5 to 9) of the intraocular lens 111. When the intraocular lens 111 is placed in the retaining cell 200, it is preferably aligned with the opening 212 in the back wall 202 so as to provide the user with an uninterrupted field of view (the aligning of the aperture of the intraocular lens with the opening in the retaining cell directs light toward the retina of the eye). In an embodiment, it is desirable for the central axis of the aperture to coincide with the central axis of the opening in the retaining cell 200.

The back wall 202 has an outer diameter of up to 10 millimeters, preferably 7 millimeter to 9.6 millimeters. The back wall 202 has a thickness of up to 0.1 millimeter and contacts the side wall 204. The entire outer circumference of the side wall 204 contacts the entire outer circumference of the back wall 204. In other words, the outer diameter of the side wall is equal to the outer diameter of the back wall. The side wall has an inner diameter of up to 8.5 millimeters, preferably 6.5 millimeters to 8.4 millimeters, and more preferably 7.0 millimeters to 8.0 millimeters.

The lip 206 protrudes from the inner surface of the side wall and extends from the inner surface of the side wall 204 toward the center of the retaining cell 200 (i.e., it protrudes in the radial direction from the inner surface of the side wall). The lip 206 protrudes towards the center from the side wall 204 for a distance of about 25 to 40% of the outer diameter of the back wall 202. The lip 206 extends towards the center in a parallel arrangement with the back wall 202 to create a space 208 between the back wall and the lip that is used to locate the intraocular lens 111 during its installation and retention in the lens capsule.

While the lip 206 shown in the FIG. 12 is bi-lobal, i.e., each lip contains two protrusions (lobes) with a slight dip in between, other geometries such as for example a triangle, a semi-circle, a square, a polygon, or the like may be used.

The retaining cell 200 is manufactured from a flexible material that is biocompatible. Elastomers are suitable examples of materials that may be used in manufacturing the retaining cell. The flexible material does not always have to be elastomeric. Biocompatible elastomers include polysiloxane-containing materials and fluoro-containing polymers. Polydimethylsiloxane and polydimethylsiloxane containing copolymers (listed above) may be used to manufacture the retaining cell 200. Examples of fluoro-containing polymers are polytetrafluoroethylene, polyvinylfluoride, polyvinylidene fluoride, polychlorotrifluoroethylene, perfluoroalkoxy polymer, fluorinated ethylene-propylene, polyethylenetetrafluoroethylene, polyethylenechlorotrifluoroethylene, perfluoropolyether, or the like, or a combination thereof.

FIG. 13 depicts one exemplary embodiment, where the intraocular lens 111 is located in the retaining cell 200. As may be seen in the FIG. 13, the fixation members 115, 117 are restrained one or more of the lips 206. In an embodiment, each fixation member may be restrained by a single lip. In an embodiment, at least two lips 206 may be used to restrain the fixation members 115 and 117 of the intraocular lens 111.

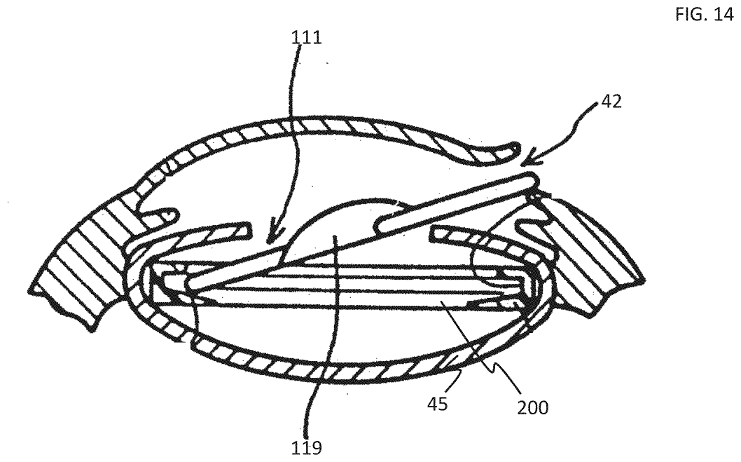

FIG. 14 depicts one exemplary embodiment of using the retaining cell 200. Firstly, the retaining cell 200 is inserted into the lenticular capsule 300. Secondly, the intraocular lens 111 is inserted into the retaining cell 200 whilst in the lenticular capsule. At this time, one of the fixation members (115 and 117) of the intraocular lens is contacted with the inner surface of the back wall 13. Then the fixation members (115 and 117) moves along the inner surface of the back wall due to the resilient restoring force thereof and extends to the space 208 so that the fixation member is completely contained in the space 208. At least one of the fixation members (115 and 117) is moved along the inner surface of the back wall towards at least one of the lips so that the fixation member lies in the space 208 completely behind the lip. The other fixation member is moved into the space 208 and takes its position partially behind one of the other lips.

In other words, the back wall 202 which extends toward a center of the retaining cell 200 acts to guide the fixation member of the intraocular lens 111 so that the intraocular lens can be easily and surely inserted and securely held. Since the back wall 202 is centripetally protruded, an ophthalmologist who performs an operation can not only confirm the back wall 202 of the device which is inserted into an eye and set but also insert and fix the intraocular lens easily and surely. The retaining cell can be retained in the lenticular capsule, when an old intraocular lens is removed and replaced with a new intraocular lens.

In another method of using the retaining cell 200, the intraocular lens 111 is inserted into the retaining cell 200 whilst outside the lenticular capsule. The retaining cell 200 with the intraocular lens 111 is then inserted into the lenticular capsule 300 through the opening 42.

It is to be noted that all ranges detailed herein include the endpoints. Numerical values from different ranges are combinable.

The transition term comprising encompasses the transition terms “consisting of” and “consisting essentially of”.

The term “and/or” includes both “and” as well as “or”. For example, “A and/or B” is interpreted to be A, B, or A and B.

While the invention has been described with reference to some embodiments, it will be understood by those skilled in the art that various changes may be made and equivalents may be substituted for elements thereof without departing from the scope of the invention. In addition, many modifications may be made to adapt a particular situation or material to the teachings of the invention without departing from essential scope thereof. Therefore, it is intended that the invention not be limited to the particular embodiments disclosed as the best mode contemplated for carrying out this invention, but that the invention will include all embodiments falling within the scope of the appended claims.

1. An intraocular lens implantable in an eye comprising:
an optical portion adapted for placement in the lens capsule of the eye and for directing light toward the retina of the eye;
an annular ring having an outer diameter and an inner diameter; and
at least one elongated fixation member coupled to said optical portion for use in fixing said intraocular lens in the eye; where the outer diameter of the annular ring contacts the elongated fixation member and where the inner diameter of the annular ring contacts the optical portion of the intraocular lens; and where the entire intraocular lens is a single monolithic piece.

2. The intraocular lens of claim 1 where the intraocular lens comprises at least two elongated fixation members that have an outer diameter of 13.5 millimeters or less.

3. The intraocular lens of claim 1, where the outer diameter of the annular ring is 7 millimeters or less.

4. The intraocular lens of claim 1, where the inner diameter of the annular ring is 6 millimeters or less.

5. The intraocular lens of claim 1, further comprising a membrane that contacts the inner diameter of the annular ring and an outer diameter of the optical portion.

6. The intraocular lens of claim 1, where the entire intraocular lens comprises a single material.

7. The intraocular lens of claim 1, where the intraocular lens comprises an acrylate.

8. The intraocular lens of claim 1, where the intraocular lens comprises a polysiloxane.

9. The intraocular lens of claim 1, where the optical portion comprises a cell barrier for preventing cell migration during or after surgery.

10. The intraocular lens of claim 1, where the optical portion is 5.5 millimeters or less in diameter.

11. A method comprising:
molding in one piece an intraocular lens comprising:
an optical portion adapted for placement in the lens capsule of the eye and for directing light toward the retina of the eye;
an annular ring having an outer diameter and an inner diameter; and
at least one elongated fixation member coupled to said optical portion for use in fixing said intraocular lens in the eye; where the outer diameter of the annular ring contacts the elongated fixation member and where the inner diameter of the annular ring contacts the optical portion of the intraocular lens; and where the entire intraocular lens is a single monolithic piece.

12. The method of claim 10, where the molding comprises injection molding.

13. A method comprising:
making an incision is made in an edge of a cornea of any eye;
making a circular opening in an anterior portion of a lens capsule of the eye;
disposing an intraocular lens within the lens capsule through the circular opening in the anterior portion; where the intraocular lens comprises:
an optical portion adapted for placement in the lens capsule of the eye and for directing light toward the retina of the eye;
an annular ring having an outer diameter and an inner diameter; and
at least one elongated fixation member coupled to said optical portion for use in fixing said intraocular lens in the eye; where the outer diameter of the annular ring contacts the elongated fixation member and where the inner diameter of the annular ring contacts the optical portion of the intraocular lens; and where the entire intraocular lens is a single monolithic piece.

14. A retaining cell for holding an intraocular lens comprising:
a back wall having an opening for accommodating an aperture of an intraocular lens; such that the opening and the aperture are aligned for directing light toward the retina of the eye;
a sidewall that contacts the back wall along its circumference;
at least two lips that contact the side wall and protrude radially from a circumference of the side wall towards a center of the opening in the back wall; where a surface of the lip is parallel to a surface of the back wall creating a space therebetween for reversibly accepting an intraocular lens.

15. The retaining cell of claim 14, further comprising an intraocular lens implantable in an eye comprising:
an optical portion adapted for placement in the lens capsule of the eye and for directing light toward the retina of the eye;
an annular ring having an outer diameter and an inner diameter; and
at least one elongated fixation member coupled to said optical portion for use in fixing said intraocular lens in the eye; where the outer diameter of the annular ring contacts the elongated fixation member and where the inner diameter of the annular ring contacts the optical portion of the intraocular lens; and where the entire intraocular lens is a single monolithic piece; where the elongated fixation member is restrained between a lip and the back wall of the retaining cell.
